(19) United States
(12) Patent Application Publication
(10) Pub. No.: US 2009/0247827 A1
(43) Pub. Date: Oct. 1, 2009

(54) Endoscope Gripping Device

(75) Inventors: Dean J. Secrest, Concord, OH (US); Christopher J. Kaye, Concord, OH (US); Aaron Boyce, Eastlake, OH (US); John P. Winstanley, Madison, OH (US)

Correspondence Address: CALFEE HALTER & GRISWOLD, LLP, 800 SUPERIOR AVENUE, SUITE 1400, CLEVELAND, OH, 44114, US

(73) Assignee: U.S. ENDOSCOPY GROUP, INC., Mentor, OH (US)

(21) Appl. No.: 12/412,850

(22) Filed: Mar. 27, 2009

Related U.S. Application Data
(60) Provisional application No. 61/039,950, filed on Mar. 27, 2008.

(51) Int. Cl. A61B 1/00

(52) U.S. Cl. 600/131

(57) ABSTRACT

A device and method for facilitating the insertion, manipulation and removal of an endoscopic medical device within a body cavity is disclosed. The device may comprise a body portion and a lining. The body portion may be configured to wrap around the outer surface of the insertion tube. The body portion may comprise an outer surface, an inner surface, two ends, and a longitudinal slit extending from one end of the body portion to the other end. The lining may be attached to the interior surface of the body portion. The lining may comprise an at least partially raised surface that contacts the outer surface of the insertion tube at least when a user squeezes the body portion of the device.

### CROSS-REFERENCE TO RELATED APPLICATIONS

This non-provisional application claims the benefit of U.S. Provisional Patent Application No. 61/039,950 entitled “Endoscope Gripping Device,” filed Mar. 27, 2008, which is hereby incorporated in its entirety to the extent it is not conflicting with the present application.

### FIELD OF THE INVENTION

The invention relates to a non-invasive device and method for facilitating the control of an endoscopic medical device which is partially inserted into a body cavity.

### BACKGROUND OF THE INVENTION

Endoscopic devices, such as endoscopes, are well-known in the art and are commonly used in numerous medical procedures. For example, an endoscope may be used to examine the gastrointestinal tract of a patient during the removal of polyps, lesions or other types of targeted tissue from the mucosal wall. During certain procedures, the examination of internal body cavities requires that the endoscope be inserted through a body orifice. Insertion of an endoscopic device through a body orifice generally requires the use of a lubricant, such as a water based lubricant.

Users of endoscopic devices find it difficult to effectively control and manipulate the device due to the layer of lubricant. For example, during the insertion or removal of the device, the user's gloved hand may slip along the outer surface of the device. This slippage affects the user's control of the device and re-distributes the layer of lubricant. In one conventional technique, a user will use sanitary towels to grip the endoscopic device and facilitate its manipulation. However, these towels become saturated with lubricant and slip along the outer surface of the device. Further, the towels may remove the layer of lubricant, causing increased distress or pain to the patient. While removal of excess lubricant is desirable, a layer of lubricant is generally required to prevent distress to the patient. Also, users of endoscopic devices employing conventional techniques will often have to look away from the internal display monitor to facilitate the manipulation of the endoscopic device. For example, during endoscopic procedures, the sanitary towels often fall off the endoscopic device forcing the user to stop the procedure and look away from the monitor to secure a replacement towel. Further, users of endoscopic devices often suffer injuries, such as for example, tendonitis, due to the stress and fatigue on the hand and arm muscle structures from twisting and positioning the endoscopic device during long, challenging therapeutic endoscopy procedures (e.g., inserting the endoscopic device through the sigmoid flexure in the large intestine).

### SUMMARY OF THE INVENTION

The invention is directed to a non-invasive device and method for facilitating the insertion and removal of an endoscopic medical device into a body cavity while maintaining lubricant on the outer surface of the endoscopic device. In an aspect of the present invention, a device for gripping the outer surface of an insertion tube of an endoscope is disclosed. The device may comprise a body portion and a lining. The body portion may be configured to wrap around the outer surface of the insertion tube. The body portion may comprise an outer surface, an inner surface, two ends, and a longitudinal slit extending from one end of the body portion to the other end. The lining may be attached to the interior surface of the body portion. The lining may comprise an at least partially raised surface that contacts the outer surface of the insertion tube at least when a user squeezes the body portion of the device. In another aspect of the present invention, a method for facilitating the insertion and manipulation of an endoscope into the body cavity of a patient is disclosed.

The invention may take form in various components and arrangements of components, and in various process operations and arrangements of process operations. Further, features and advantages of this invention will become apparent from the following detailed description made with reference to the accompanying drawings.

### BRIEF DESCRIPTION OF THE DRAWINGS

The drawings are only for the purpose of illustrating preferred embodiments and are not to be construed as limiting the invention.

FIGS. 1 and 2 are perspective views of a device according to an embodiment of the invention, wherein the device is attached to an insertion tube of an endoscope;

FIG. 3A is a perspective view of the device of FIGS. 1 and 2;

FIG. 3B is an end view of the device of FIGS. 1 and 2;

FIG. 3C is a cross sectional view of the device of FIGS. 1 and 2 along the line 3C-3C in FIG. 3B;

FIG. 4 is a perspective view of the device of FIGS. 1 and 2, wherein the device is in an open configuration to show an inner surface and a lining of the device;

FIG. 5 is a perspective view of a device according to another embodiment of the invention, wherein the device is in an open configuration to show an inner surface and a lining of the device;

FIGS. 6A and 6B are perspective views of a device according to another embodiment of the invention;

FIG. 6C is an end view of the device shown in FIGS. 6A and 6B;

FIG. 7A is a perspective view of a device according to another embodiment of the invention;

FIG. 7B is a side view of the device shown in FIG. 7A; and

FIG. 7C is an end view of the device shown in FIG. 7A.

### DETAILED DESCRIPTION OF THE INVENTION

This Detailed Description of the Invention merely describes embodiments of the invention and is not intended to limit the scope of the specification in any way. Indeed, the invention as described by the specification is broader than and unlimited by the preferred embodiments, and the terms used in the specification have their full ordinary meaning.

The invention will be described for use with an endoscopic device. More specifically, the invention will be described for use with the insertion tube portion of an endoscope. It should be understood, however, that this is for exemplary purposes only and the invention can be applied to a wide variety of applications.

A device and method of use for gripping an endoscope is disclosed. The device allows the user to effectively manipulate the endoscope while maintaining a layer of lubricant on the endoscope and reducing the user fatigue associated with manipulating the endoscope, e.g., during introduction of the endoscope or while applying torque on the endoscope. Further, a simple, easy to use device is disclosed that allows the user to effectively manipulate an endoscope without looking away from the monitor. The device may also direct the lubricant somewhere other than the gripping surface of the device, keeping the user's gloved hand dry and free from lubricant.

The device may be made from any suitable material known in the art capable of gripping an endoscope, such as a thermoplastic elastomer (“TPE”), plastic, foam, silicone, or sponge material. For example, in one embodiment, the body portion of the device is made from Nitrile Butadiene Rubber (NBR) Foam. The material of the device may be absorptive (i.e., capable of absorbing excess lubricant). The device may also be soft or hard (e.g., various materials having various thicknesses). A soft device cushions the user's hand and generally provides for a more comfortable fit than a hard device. The device may also be stiff or flexible (e.g., materials having varying durometers). Further, the device may be made by any suitable manufacturing method known in the art, such as molding.

The device may be sized to accommodate endoscopes of varying outer diameters, for example, to accommodate pediatric and adult applications. Further, the thickness and outer diameter of the device, i.e., the diameter of the outer gripping surface, may vary. Generally, a soft, thin device allows the user to better “feel” the endoscope than a thicker device. An optimal outer diameter range, for example 0.60-1.75 inches, provides a high level of ergonomic comfort and control of the endoscope, while still allowing the user to feel any resistance of the intubation. An optimal diameter of the outer surface may also depend on the user's hand size. Further, the thickness and outer diameter of the device may vary along the length of the device.

The device may self release from the endoscope. In other words, when the user releases the device (i.e., removes the gripping force), the device will “spring open” such that the user may slide the device along the outer surface of the endoscope without first applying a force to release the device from the endoscope. Various sizes (e.g., inner and outer diameters) and various materials may be used such that the device “springs open” while still maintaining its position on the endoscope (i.e., without falling off or sliding down the endoscope). For example, the device may be made from a material having memory capabilities. The memory material may be encapsulated in another material, such as TPE, plastic, foam, or a sponge-like material. When in use, the memory capabilities of the material will serve to maintain the positioning of the device and prevent the device from coming off the endoscope.

The device generally includes an outer surface, an inner surface, and two ends. The outer surface of the device may include an ergonomic contour shaped to fit the user's hand and fingers. The contour may be simple or complex. For example, the outer surface may include features, such as raised portions, ridges, cavities, troughs, channels, nubs, or grooves, which improve the user's grip on the device and provide a more comfortable fit and feel. The raised features on the outer surface may also act as a lever providing the user with a mechanical advantage when manipulating the endoscope. Further, the outer surface may include raised portions, such as flanges, on either end of the outer surface to prevent the user's hand from slipping off the device when in use. These raised portions also may allow the user to determine the extents of the device without looking away from the monitor. Further, the raised portions may help reduce the amount of gripping force required to manipulate the endoscope and act as a shield to prevent excess lubricant from contacting the user's hand. The outer surface may also include contoured portions designed to fit the user's thumb and fingers, allowing for enhanced control of the device and providing the user with a mechanical advantage when manipulating the endoscope.

The device may include features that permit at least a portion of the user's hand or fingers to contact the outer surface of the endoscope when the device is in use. These features allow the user to grip the endoscope directly to effectively manipulate the endoscope. For example, the device may include openings, such as notches, extending through the device from the outer surface to the inner surface. The openings are configured to allow the user's fingers to contact and grip the outer surface of the endoscope when the device is attached to, or wrapped around, the endoscope.

The device may also include a strap to secure the device to the user's hand. The strap is intended to prevent the user's hand from slipping off the device when in use. Further, the strap prevents the device from falling to the floor when not in use. As such, the user will not have to stop the procedure and look away from the internal display monitor to locate the device or secure a replacement device. The strap may be attached to the device in a variety of ways. For example, each end of the strap may be attached to an end of the device, or raised portion thereof, such that the strap extends across the outer surface of the device from one end of the device to the other. As such, the strap would contact the outer portion of the user's hand or fingers when the user is gripping the device. The strap may be made from the same material of the device body and/or a variety of materials know in the art, for example rubber or foam.

The device may include a clip, or other similar fastener, configured to hold the device on the endoscope. For example, the clip may be shaped and configured to snap around the endoscope and/or the body portion of the device (e.g., at one or both ends of the device) to hold the device in place on the endoscope. The clip may also be attached or integrally formed with the device to prohibit separation of the clip from the device. The clip may be made from various resilient materials, such as steel, plastic, or any other like material.

The device may also include interlocks integrally molded with the device to hold the device on the endoscope. For example, one or both ends of the device may include a strap that wraps around the endoscope to hold the device on the endoscope and prohibit axial movement of the device relative to the endoscope. In one embodiment, a first end of the strap is inserted through an opening located towards a second end of the strap to fasten the device to the endoscope. In another embodiment, the first and second ends of the strap are twisted together to fasten the device to the endoscope. In these and other embodiments, the interlock may be adjustable to be used on various scopes with different diameters. The interlocks may be made from a variety of materials, such as foam, TPE, or a soft polymer.

Further, the device may include a longitudinal slit, or opening, that allows the device to attach to, or wrap around, the endoscope. The slit generally extends from one end of the device to the other, creating two longitudinal edges. The width of the slit may vary depending on numerous factors, including the size of the endoscope and the rigidity of the device. The width of the slit may also vary along its length. For example, a slit may be narrower at one end to prohibit the device from coming off the endoscope when not in use, but not too narrow such that the device may still be easily mounted on to the insertion tube portion of the endoscope. The slit may also be positioned away from the user's palm and fingers when the device is in use to provide for a more comfortable and ergonomic fit and feel.

Further, device may include a fastener, such as a belt, VELCRO®, a tension strap, or a spring loaded latch, to bridge the gap between the longitudinal edges of the slit, prohibiting the device from coming off the endoscope. For example, a tension strap may be used to bias the longitudinal edges of the slit together. The tension strap may be at least partially removed or stretched to allow the device to attach to, or wrap around, the endoscope. Further, buttons, or knobs, on the outer surface of the device may control a locking mechanism, such as a latch. The locking mechanism may be biased toward a closed position, such as with a spring, preventing the device from coming off the endoscope. The buttons, or knobs, may be used to open the locking mechanism and allow the device to attach to, or wrap around, the endoscope. Further, the fastener may be used for devices that accommodate endoscopes of varying outer diameters (i.e., “one size fits all” devices).

The inner surface of the device contacts the outer surface of the endoscope. The inner surface may be contoured to facilitate the effective manipulation of the endoscope while maintaining a layer of lubricant on the endoscope. For example, the inner surface may include raised portions, such as ridges or dots, which contact the outer surface of the endoscope as the user squeezes the device, allowing the user to grip the endoscope. These raised portions may form various patterns on the inner surface, such as straight lines, curved lines, dotted lines, mesh, or crossed lines. Further, these raised portions form channels, or troughs, for the lubricant. The channels allow the lubricant to flow between the device and endoscope to maintain the layer of lubricant on endoscope. The pattern of raised portions may be included on the inner surface by any suitable method known in the art. For example, the pattern may be molded or overmolded, or even attached, such as with an adhesive, to the inner surface. Further, the memory material discussed above may also serve as the inner surface or contour of the device. For example, coated wire having a mesh pattern may be included on the inner surface to facilitate the effective manipulation of the endoscope while maintaining a layer of lubricant on the endoscope.

For example, in one embodiment, the device includes a mesh lining, such as a needle point canvas, attached to the inner surface of the device. The lining may be attached to the inner surface by any suitable means, such as with a double coated polyethylene tape, transfer tape, hot melt, epoxy, ultraviolet curable adhesive, or other suitable adhesive. The lining may cover the entire inner surface of the device or only a portion of the inner surface. The pattern of the mesh lining displaces the lubricant on the outer surface of the endoscope to facilitate gripping of the endoscope by the device. In another embodiment, the lining comprises a non-woven polyester tape attached to the inner surface of the device. The tape lining may be attached to the inner surface by any suitable means, such as those discussed above. The tape lining may also comprise a pattern that displaces the lubricant on the outer surface of the endoscope to facilitate gripping of the endoscope by the device. In these and other embodiments, the hardness (e.g., durometer) of the lining may affect the ability of the device to grip the endoscope. For example, a hard lining may provide a better grip on the endoscope than a softer lining.

The cross sectional shape of the device may be a variety of shapes known in the art suitable for manipulating the endoscope while maintaining a layer of lubricant on the endoscope, for example, C-shaped or polygonal shaped, such as diamond-shaped or pentagon-shaped. Depending on the cross sectional shape of the device, more or less area of the inner surface of the device may contact the outer surface of the endoscope. For example, generally, a C-shaped device will provide more contact area than a diamond-shaped device. However, as a general rule, as the contact area increases, the volume of lubricant allowed to flow between the device and the endoscope decreases. As such, various combinations of cross sectional shapes and inner surface contours may be used to balance the amount of contact area required to effectively manipulate the endoscope with the volume of lubricant flow between the device and the endoscope.

The device may also include features that assist with the removal of excess lubricant from the outer surface of the endoscope. For example, one or more pieces of cloth, or fabric, may be attached to the device, such as to the inner surface or ends of the device, to collect the excess lubricant as the device moves along or is squeezed against the outer surface of the endoscope. The cloth or fabric may include raised portions or channels that facilitate the effective manipulation of the endoscope while maintaining a layer of lubricant on the endoscope. In certain embodiments, the device includes a TPE body that provides increased diameter structure with enlarged ends for sliding the device along the endoscope without looking down and an internal cloth provides the lubrication displacement qualities needed for the grip potential on the insertion tube portion of the endoscope.

Further, the device may include reservoirs that collect excess lubricant from the outer surface of the endoscope. The reservoirs may be a variety of shapes and sizes. For example, in certain embodiments, the reservoirs are cut in to the inner surface of the device. However, the reservoirs may also be cut in to the outer surface of the device. For example, the reservoirs may be cut into either end of the outer surface to collect excess lubricant as the user moves the device along the outer surface of the endoscope. In some embodiments, the reservoirs include notches that extend between the outer surface and inner surface of the device. The shape, size, and angle of the notches help to channel the flow of excess lubricant from the outer surface of the endoscope and into the notches, or reservoirs. In these embodiments, a cover, or membrane, may be used to prevent the excess lubricant from escaping to the outer surface of the device. Further, raised portions and channels on the inner surface of the device may be used to direct the flow of the excess lubricant in to the reservoirs. Absorptive material, such as one or more pieces of a cloth or towel, may also be used to absorb any excess lubricant. Finally, the device may direct the flow of excess lubricant to the reservoirs regardless of which direction the device is moved along the surface of the endoscope.

Further, the network of channels formed from the raised portions on the inner surface of the device may be accessed by a suction tube, or line. For example, the network of channels may resemble a maze that leads to a main suction tube. As such, excess lubricant from the outer surface of the endoscope may flow through the network of channels to the suction tube. The suction would pull the excess lubricant through the tube and away from the inner or gripping surface of the device when the device is surrounding the endoscope insertion tube. The suction tube may be attached at any location on the perimeter of the device to access the network of channels. In certain embodiments, for example those embodiments in which the device is made from foam, the outer surface of the device may require a non permeable skin coating to focus the suction so that it draws the excess lubricant away from the inner or gripping surface of the device.

The suction tube may also be attached to the reservoirs, or troughs, of the device that collect excess lubricant from the outer surface of the endoscope. For example, reservoirs may be cut into either end of the device such that the excess lubricant collects in the reservoirs as the user slides the device along the outer surface of the endoscope. As such, the suction would pull the excess lubricant through the tube and away from the outer surface of the endoscope.

Suction may also be used to hold the device relative to the endoscope. For example, the device may be configured such that a suction tube may access a network of device channels that open on the inner surface and/or longitudinal edges of the device. As suction is applied through the channels, the device would clamp down on the outer surface of the endoscope to prohibit movement of the device relative to the endoscope. When the suction is removed, the device would release and be able to move relative to the endoscope. The suction may be controlled using a valve, such as a manually operated foot pedal or switch.

In certain embodiments, the device includes a piece of flexible material, such as a TPE, having a contoured surface similar to the raised portions on the inner surface described above. For example, the device may be in the shape of rectangular sheet having a contoured surface on one or both sides. The user wraps the sheet around the endoscope such that the contoured surface contacts the outer surface of the endoscope. Further, the device may be in the shape of a glove having a contoured surface. As discussed earlier, the raised portions of the contoured surface facilitate the effective manipulation of the endoscope while maintaining a layer of lubricant on the endoscope.

One exemplary method of using the device includes the step of inserting the endoscope through the slit, or opening, such that the device wraps around the endoscope. Thus, the device may wrap around the endoscope after the endoscope is inserted into the body. However, the endoscope may also be inserted through the ends of the device prior to the insertion of the endoscope into the body (i.e., end-loaded). In some embodiments, the user may use a fastener to prevent the device from coming off of the endoscope. Next, the device may be positioned on the endoscope by sliding it along the outer surface of the endoscope. As discussed above, the device maintains the layer of lubricant on the endoscope as it slides along the outer surface of the endoscope. In certain embodiments, the device removes excess lubricant from the outer surface of the endoscope as it slides along the outer surface. After the device is positioned, the user may then squeeze the device such that the inner surface, or portions thereof, grip the outer surface of the endoscope. At this point, the device and the endoscope will move together and the user may effectively manipulate the endoscope. Finally, to reposition the device on the endoscope, the user will release the device and slide it along the outer surface of the endoscope.

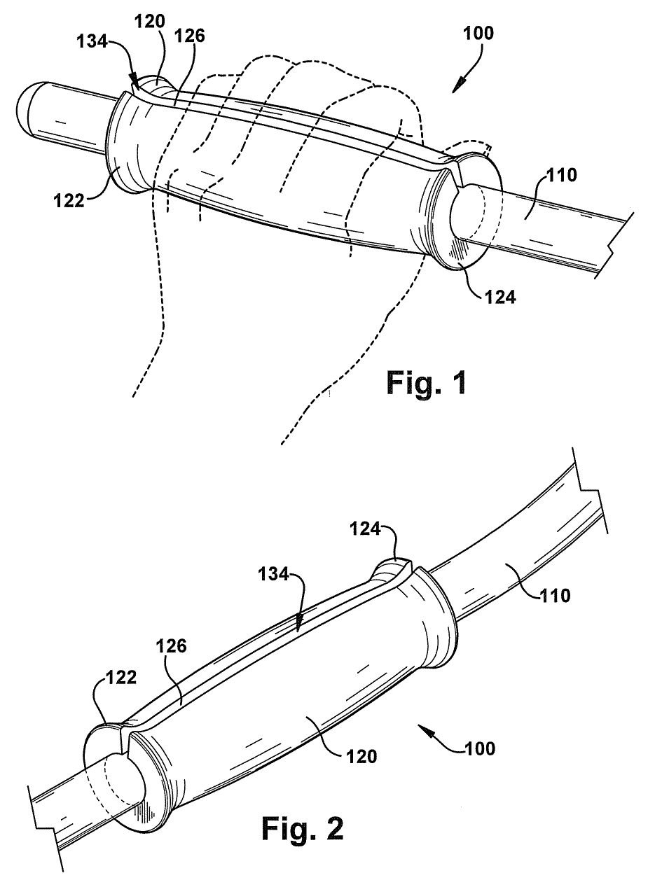

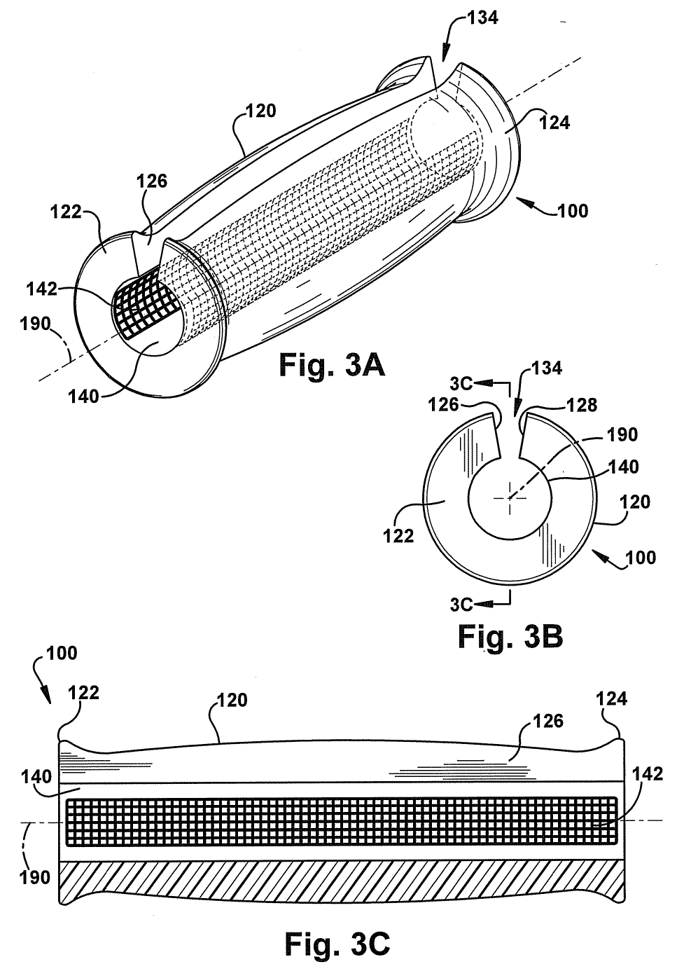

Referring now to the drawings, FIGS. 1-4 illustrate a device 100 according to an embodiment of the invention. The exemplary device 100 depicted in the figures includes a body portion and a lining 142. The body portion includes an outer surface 120, an inner surface 140, two ends 122, 124, and a longitudinal slit 134. A longitudinal axis 190 of the device 100 is depicted in FIGS. 3A-3C. The body portion of the device 100 is made from NBR Foam. The lining 142 of the device 100 is a mesh needle point canvas and can be made from combed cotton canvas and manufactured by MCG Textiles (12 mesh), but may be manufactured from other materials, such as plastic or TPE.

FIGS. 1 and 2 illustrate the exemplary device 100 in use and attached to, or wrapped around, the insertion tube 110 of an endoscope. FIG. 1 schematically depicts a user's hand in a position for using the device 100. The device 100, as illustrated, is symmetric to facilitate use by either a user's right hand or left hand, among other reasons. The insertion tube 110 may be inserted through the slit 134 such that the device 100 wraps around the tube. Thus, the device 100 may wrap around the insertion tube 110 after the tube is inserted into the body. Further, the device 100 may be removed from the insertion tube 110 without removing the tube from a patient. However, the insertion tube 110 may also be inserted through the ends 122, 124 of the device 100 prior to the insertion of the tube into the body (i.e., end loaded).

The outer surface 120 of the exemplary device 100 is illustrated in FIGS. 1-3C. The ergonomic contour of the outer surface 120 is shaped to fit the user's hand and provides a comfortable fit and feel for the user. The device 100 includes flanges on both ends 122, 124 of the device. The flanges prevent the user's hand from slipping off the device 100 when in use and also act as a shield to prevent excess lubricant from contacting the user's hand. Further, the flanges allow the user to determine the extents of the device 100 without looking at the device, i.e., away from the endoscope monitor.

The longitudinal slit 134 of the exemplary device 100 is illustrated in FIGS. 1-3B. The slit 134 extends from one end 122 of the device 100 to the other end 124, forming two longitudinal edges 126, 128. The slit 134 allows the device 100 to attach to, or wrap around, the insertion tube 110. As illustrated in FIG. 3B, the longitudinal edges 126, 128 of the slit 134 are angled such that the outer portion of the edges are farther apart than the inner portion of the edges. The wider gap between the portion of the edges 126, 128 towards the outer surface 120 facilitates attachment, or mounting, of the device 100 to the insertion tube 110. Further, the narrower gap between the portion of the edges 126, 128 towards the inner surface 140 prohibits the device 100 from coming off the insertion tube 110 during use. However, in other embodiments, the longitudinal edges 126, 128 are parallel to each other.

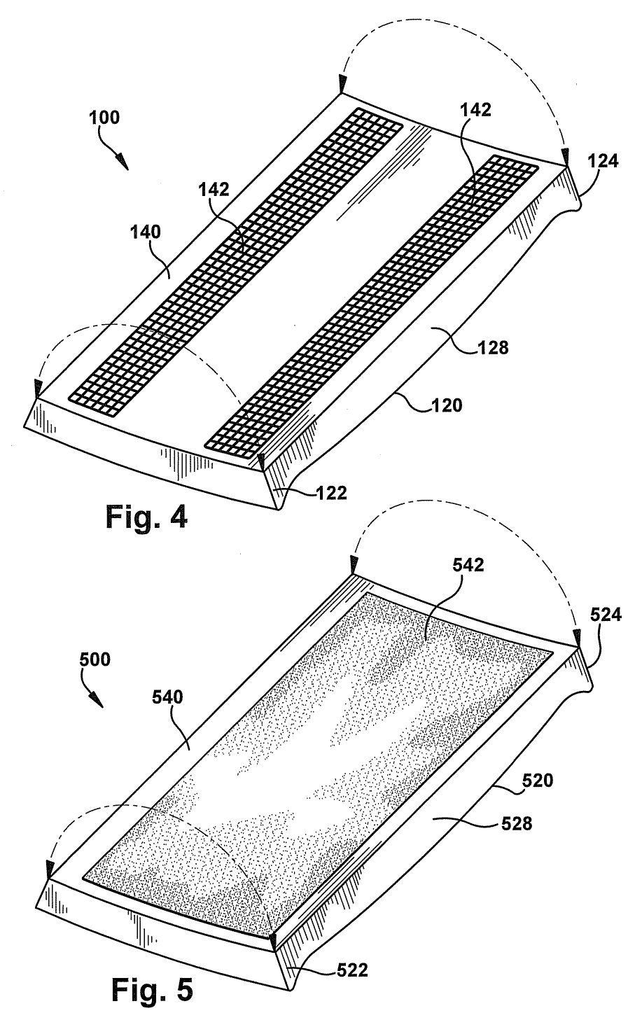

The inner surface 140 of the exemplary device 100 is illustrated in FIGS. 3A-4. As illustrated in FIG. 3B, the device 100 has a C-shaped cross section. The diameter of the inner surface 140 may be sized to accommodate insertion tubes of various sizes. Further, as illustrated in FIGS. 3A, 3C, and 4, a lining 142 is attached to the inner surface 140 of the device 100. The lining 142 may be molded, overmolded, or attached to the inner surface 140 by any suitable means, such as with a double coated tape, transfer tape, hot melt, epoxy, ultraviolet curable adhesive, or other suitable adhesive. The lining 142 may cover the entire inner surface 140 of the device 100 or only a portion of the inner surface. As shown, the lining 142 includes two mesh runners attached to the inner surface 140 of the device 100 by a double coated tape. The double coated tape may be an adhesive tape having a pressure-sensitive adhesive coated on both sides of the facestock material. The facestock material may be a thin flat film, such as polyester, polyethylene, polypropylene, or the like. In some embodiments, the lining 142 is attached to the inner surface 140 of the device 100 by a transfer tape. The transfer tape may be an adhesive tape having a layer of pressure-sensitive adhesive with no facestock or supporting film. The transfer tape is generally supplied on a release liner which is removed when the adhesive is laminated to a substrate.

The lining 142 attached to the inner surface 140 of the device 100 includes a mesh pattern of raised portions on the inner surface. The raised portions contact the outer surface of the insertion tube 110 (shown in FIGS. 1-2) as the user squeezes the device 100. The mesh pattern of raised portions displaces the lubricant on the outer surface of the insertion tube 110 to facilitate gripping of the tube by the device 100. Further, the depressions between the raised portions allow the lubricant to flow between the device 100 and the insertion tube 110 to maintain the layer of lubricant on the outer surface of the tube. The hardness (e.g., durometer) of the mesh raised portions affect the ability of the device 100 to grip the insertion tube 110. For example, a lining with hard raised portions may provide a better grip on the insertion tube 110 than a lining with softer raised portions.

FIG. 5 illustrates a device 500 according to another embodiment of the invention. The exemplary device 500 depicted includes a body portion and a lining 542. Similar to device 100, the body portion includes an outer surface 520, an inner surface 540, two ends 522, 524, and a longitudinal slit. As shown, the body portion of the device 500 is made from NBR Foam. The lining 542 of the device 500 is a single coated non-woven tape. The tape may be an adhesive tape having a pressure-sensitive adhesive coated on one side of a facestock material. The facestock material may be a flat porous sheet with a structure that includes both solid material and void areas, such as a non-woven fabric.

As stated, the body portion of device 500 is similar to the body portion of device 100 illustrated in FIGS. 1-4. However, the lining 542 is different from the lining 142 of device 100. In particular, the lining 542 attached to the inner surface 540 of the device 500 comprises a tape having a pattern of small raised portions (not shown) that contact the outer surface of the insertion tube as the user squeezes the device 500. The raised portions of the tape lining 542 displace the lubricant on the outer surface of the insertion tube to facilitate gripping of the tube by the device 500. Further, small depressions, voids, between the raised portions of the tape lining 542 allow the lubricant to flow between the device 500 and the insertion tube to maintain the layer of lubricant on the outer surface of the tube. The hardness (e.g., durometer) of the raised portions of the tape lining 542 affect the ability of the device 500 to grip the insertion tube. For example, a tape lining with hard raised portions may provide a better grip on the insertion tube than a tape lining with softer raised portions. The tape lining 542 may cover the entire inner surface 540 of the device 500 or only a portion of the inner surface. As shown, the tape lining 542 includes a single strip of tape that substantially covers the inner surface 540 of the device 500.

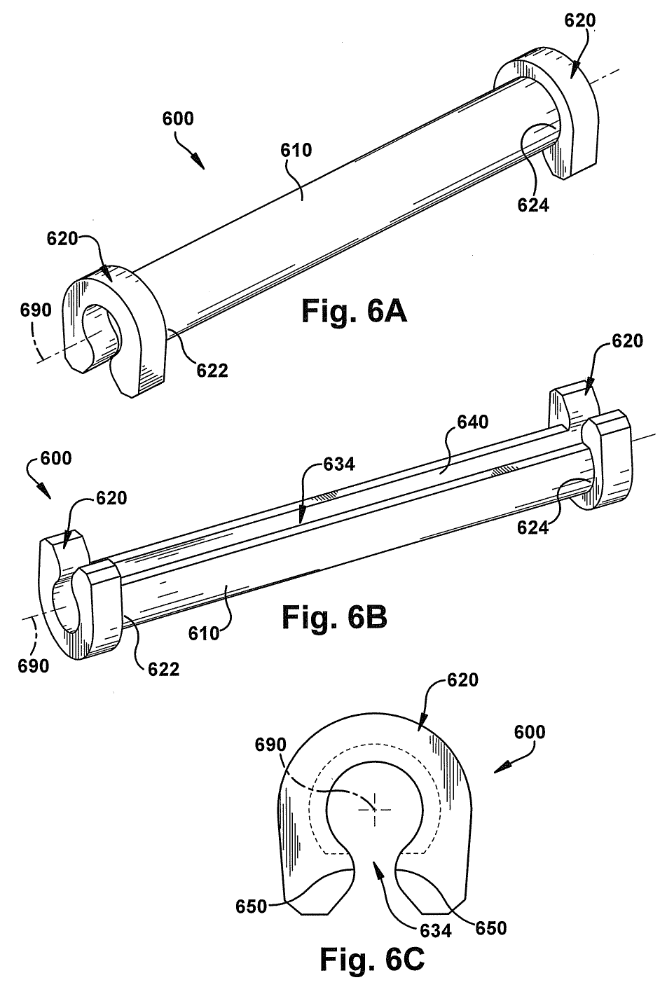

FIGS. 6A-6C illustrate a device 600 according to another embodiment of the invention. The exemplary device 600 depicted comprises a body portion and two clips 620. Similar to device 100, the body portion includes an outer surface 610, an inner surface 640, two ends 622, 624, and a longitudinal slit 634. A longitudinal axis 690 of the device 600 is shown in FIGS. 6A-6C.

The two clips 620 hold the device 600 in place on the insertion tube. The clips 620 are resilient and configured to snap around the insertion tube and/or the body portion of the device 600 to hold the device in place. With reference to FIG. 6C, each clip 620 is configured such that two raised portions 650 of the clip are biased towards each other to allow the clip to snap around, or clamp on, the insertion tube and/or body portion of the device 600. As shown, the clips 620 are positioned at each end 622, 624 of the device 600 to hold the device in place and prohibit movement of the device along the outer surface of the insertion tube. However, in other embodiments, more or less clips are used to hold the device 600 in place. Further, the clips may be positioned at various locations along the longitudinal length of the device 600. In some embodiments, one or more clips are attached or integrally formed with the device to prohibit separation of the clips from the device. The clips may be made from various resilient materials, such as steel, plastic, or any other like material.

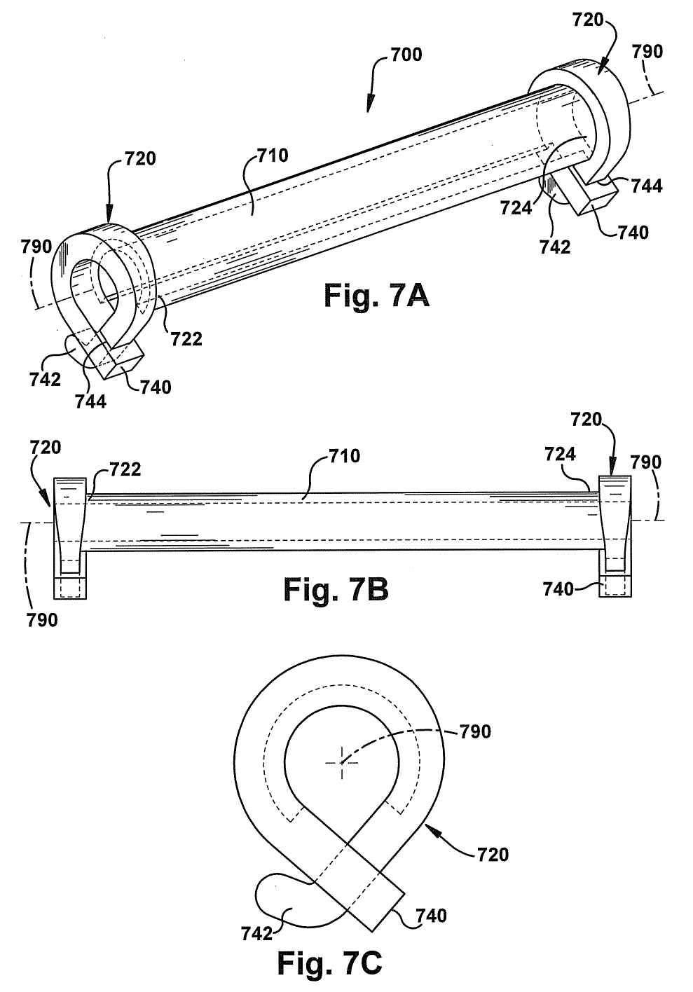

FIGS. 7A-7C illustrate a device 700 according to another embodiment of the invention. The exemplary device 700 depicted includes a body portion and two interlocks 720. Similar to device 100, the body portion includes an outer surface 710, an inner surface, two ends 722, 724, and a longitudinal slit. A longitudinal axis 790 of the device 700 is shown in FIGS. 7A-7C.

The two interlocks 720 hold the device 700 in place on the insertion tube and prohibit axial movement of the device relative to the insertion tube. As shown, the interlocks 720 are straps, or belts, integrally molded with the device 700 that wrap around the insertion tube to hold the device on the insertion tube. The first end 742 of each interlock 720 is inserted through an opening 744 located towards a second end 740 of the interlock to fasten the device 700 to the insertion tube. In another embodiment, the first 742 and second end 740 of each interlock 720 are twisted together to fasten the device 700 to the insertion tube. As shown, the interlocks 720 are positioned at each end 722, 724 of the device 700 to hold the device in place and prohibit axial movement of the device along the outer surface of the insertion tube. However, in other embodiments, more or less interlocks are used to hold the device 700 in place. Further, the interlocks may be positioned at various locations along the longitudinal length of the device 700. Each interlock 720 may be adjustable to be used on various insertion tubes with different diameters. The interlocks 720 may be made from a variety of materials, such as foam, TPE, or a soft polymer.

While several embodiments of the invention have been illustrated and described in considerable detail, the invention is not to be considered limited to the precise constructions disclosed. Various adaptations, modifications, and uses of the invention may occur to those skilled in the arts to which the invention relates. It is the intention to cover all such adaptations, modifications, and uses falling within the scope or spirit of the specification filed herewith.

We claim:

1. A device for gripping the outer surface of an insertion tube of an endoscope, comprising:
a body portion configured to wrap around the outer surface of the insertion tube, the body portion having an outer surface, an inner surface, two ends, and a longitudinal slit extending from one end of the body portion to the other end; and
a lining attached to the interior surface of the body portion, the lining having an at least partially raised surface that contacts the outer surface of the insertion tube at least when a user squeezes the body portion of the device.

2. The device of claim 1, wherein axial movement of the device is prohibited relative to the insertion tube when the user squeezes the body portion of the device, whereby the user can manipulate the insertion tube when squeezing the device.

3. The device of claim 1, wherein the device is configured to move relative to the outer surface of the insertion tube when the user is not squeezing the body portion of the device.

4. The device of claim 1, wherein the at least partially raised surface of the lining displaces lubricant on the outer surface of the insertion tube to facilitate gripping of the insertion tube by the device.

5. The device of claim 1, wherein the lining comprises depressions that allow lubricant to flow between the inner surface of the body portion and the outer surface of the insertion tube.

6. The device of claim 1, wherein the body portion of the device is made from Nitrile Butadiene Rubber Foam.

7. The device of claim 1, wherein the body portion of the device has a C-shaped cross section.

8. The device of claim 1, wherein the body portion of the device has a polygonal shaped cross section.

9. The device of claim 1, wherein at least one end of the body portion comprises a flange that prohibits the user's hand from axially slipping off the device.

10. The device of claim 1, wherein the outer surface of the body portion is contoured.

11. The device of claim 1, wherein the longitudinal slit forms two longitudinal edges angled such that the outer portion of the edges are farther apart than the inner portion of the edges.

12. The device of claim 1, wherein the lining comprises a mesh pattern.

13. The device of claim 12, wherein the lining is attached to the inner surface of the device by at least one of a double coated tape and a transfer tape.

14. The device of claim 1, wherein the lining comprises two mesh runners attached to the inner surface of the device by a double coated tape.

15. The device of claim 1, wherein the lining comprises a tape with an at least partially raised surface attached to the inner surface of the body portion.

16. The device of claim 1 further comprising at least one clip configured to hold the device in place on the insertion tube.

17. The device of claim 1, wherein the body portion comprises at least one opening extending through the device from the outer surface to the inner surface, wherein the at least one opening is configured to allow the user's fingers to contact and grip the outer surface of the insertion tube.

18. The device of claim 1 further comprising at least one interlock having a first end and a second end, wherein the first end is inserted through an opening in the second end to fasten the device to the insertion tube.

19. The device of claim 1 further comprising at least one interlock having a first end and a second end, wherein the first end and the second end are twisted together to fasten the device to the insertion tube.

20. A device for gripping the outer surface of an insertion tube of an endoscope, comprising:
a cylindrical body portion configured to wrap around the outer surface of the insertion tube, the body portion having an outer surface, an inner surface, two ends, a C-shaped cross section, and a longitudinal slit extending from one end of the body portion to the other end; and
a lining attached to the interior surface of the body portion, the lining having a portion with a raised surface that contacts the outer surface of the insertion tube at least when a user squeezes the body portion of the device and depressions that allow lubricant to flow between the inner surface of the body portion and the outer surface of the insertion tube, wherein the raised surface of the lining displaces lubricant on the outer surface of the insertion tube when a width of the slit is decreased by a user squeezing the device.

21. A method for facilitating the insertion of an insertion tube of an endoscope into the body of a patient, comprising the steps of:
providing a device having a body portion configured to wrap around the outer surface of the insertion tube, the body portion having an outer surface, an inner surface, two ends, and a longitudinal slit extending from one end of the body portion to the other end; and a lining attached to the interior surface of the body portion, the lining having an at least partially raised surface that contacts the outer surface of the insertion tube at least when a user squeezes the body portion of the device;
inserting the insertion tube through the longitudinal slit such that the device wraps around the outer surface of the insertion tube;
positioning the device on the insertion tube by sliding the device along the outer surface of the insertion tube; and
squeezing the device such that the at least partially raised surface of the lining grips the outer surface of the insertion tube.

22. The method of claim 21 further comprising inserting the insertion tube into the body of a patient.

23. The method of claim 21 further comprising releasing the device from a first location and sliding the device along the outer surface of the insertion tube to re-position the device on the insertion tube at a second location.

24. The method of claim 21, wherein the insertion tube is inserted through at least one end of the device prior to insertion of the insertion tube into the body of a patient.

25. The method of claim 21, wherein the device and the insertion tube move axially together when the user squeezes the body portion of the device such that the user can manipulate the insertion tube.

26. The method of claim 21, wherein the device is configured to move relative to the outer surface of the insertion tube when the user is not squeezing the body portion of the device.

27. The method of claim 21, wherein the at least partially raised surface of the lining displaces lubricant on the outer surface of the insertion tube to facilitate gripping of the insertion tube by the device.
